(19) United States
(12) Patent Application Publication
(10) Pub. No.: US 2012/0067293 A1
(43) Pub. Date: Mar. 22, 2012

(54) Biofiltration Applicator for Controlling and Accessing the Initial Point of Contact

(76) Inventors: ROGER H. TRELOAR, SOLON, IA (US); ROBERT R. TRELOAR, FOUNTAIN HILLS, AZ (US)

(21) Appl. No.: 12/823,878

(22) Filed: Jun. 25, 2010

Related U.S. Application Data
(60) Provisional application No. 61/221,314, filed on Jun. 29, 2009.
(60) Provisional application No. 61/347,879, filed on May 25, 2010.

(51) Int. Cl. F24F 7/007; C12M 1/12

(52) U.S. Cl. 119/448; 435/283.1

(57) ABSTRACT

A woodchip based bio-filtration system for odor and particulate matter removal from exhaust fan emissions from confined animal feeding operation buildings where visual inspection of the initial point of contact between the airflow from the building and the organic media can be made, and where the initial point of contact is directly sprayed with nozzles and/or stirred or agitated manually or mechanically to disperse matter (feathers, dust, fecal matter, etc.) which might tend to restrict airflow through the media.

### BACKGROUND OF THE INVENTION

The current direction of public sentiment is to improve air quality in the communities that they reside. With population growth and a higher density of people throughout the country, a growing number of air quality complaints have occurred. The agricultural sector probably receives the highest number of these complaints due to current production practices. Confined Animal Feeding Operations (CAFO's), where a large number of animals are housed in a small area, create a significant amount of animal feces and waste. Commonly, this waste is stored in a pit directly below the animals. To have proper air quality within the production facility for the animals, exhaust fans must pull fresh air from the outside into the building while exhausting internal odorous air. These exhaust emissions contain ammonia, hydrogen sulfide, particulate matter or dust and many other unwanted pollutants. The Environmental Protection Agency (EPA) has recently conducted a study to determine the amounts of these pollutants that are created by different species, such as poultry, dairy, swine, beef and turkey. Allowable emission levels will be determined in the very near future.

To provide a remedy for odor issues, many air quality technologies have evolved. Probably the most researched has been biofiltration. Take a layer of organic media and pass air through it and you have a process called biofiltration. Activate this layer with a liquid such as water and you create an active microbial community. The bacteria within this community can then digest the particulate matter and reduce the concentration of unwanted gases.

Commonly, biofiltration systems are implemented by placing wood pallets on the ground and covering them with a porous material such as snow fencing or netting creating a support floor. The media, usually wood chips, is placed over this support floor. The exhaust emissions from the production facility are enclosed by a duct that diverts the air flow into the area below the support floor and then up through the media. The University of Minnesota, South Dakota State University, Iowa State University, Purdue University to name a few have formulated design specifications for sizing these systems.

However, these systems often have two major reoccurring problems. The first is a buildup of particulate matter in the media that causes clogging. The second is the media becoming anaerobic in certain areas from too much moisture.

In addressing the first problem, feathers, dust and airborne fecal matter often first come in contact with the filtering media at the porous netting. This netting may be beneath one to two feet of media. As these airborne pollutants enter the filtering media, they tend to overwhelm the bacteria at the initial point of contact (IPC) and cause coating or clogging of the media that restricts airflow that can endanger the animals housed within the CAFO. To fix such problems the media must be mixed to clean the IPC. The cleaning is achieved by removing the media and mixing the buildup of particulate matter with the rest of the media and then placing it back upon the support floor. This process is very labor intensive and time consuming.

The second problem with biofiltration is the maintenance of moisture levels within the media. It is generally accepted that the media moisture level should be between thirty and sixty percent to maximize the efficiency of the system. Current systems designed by the previously mentioned universities use soaker hoses or lawn sprinklers to apply water to the biofilter. Excessive rainfall or high winds can unevenly distribute water causing pockets of media to become too wet. If this occurs the media may become anaerobic and actually produce odors.

### SUMMARY OF THE INVENTION

It is an advantage of the invention to contain biofiltration media in different ways to allow human access to the initial point of contact (IPC) between the airflow and the media.

It is an advantage of the invention to have an access door to allow human access to the IPC, to simplify maintenance and cleaning and allow visual inspection of the media.

It is an advantage of the invention to have spray nozzles installed in such a way that a liquid such as water can be evenly sprayed onto the media.

It is an advantage of the invention to have spray nozzles that apply a liquid such as water to the IPC.

It is an advantage of the invention to have human access to the IPC to manually or mechanically scrap, rake, brush or mix the IPC.

It is a feature of this invention to have a pressure sensor to measure ventilation efficiency.

It is another feature of this invention to have the pressure sensor connected to a warning light or other trigger mechanism.

It is yet another advantage of this invention to have a mechanical rake which is activated by the pressure sensor to automatically stir the media.

It is an advantage of this invention to be modular in design to allow for multiple installations.

It is an advantage that the IPC is covered with a transparent material to allow sunlight in.

It is an advantage that the IPC is covered to control rainfall and wind and better maintain the system.

It is an advantage of this invention to be significantly smaller than conventional biofiltration systems.

### BRIEF DESCRIPTION OF DRAWINGS

FIG. 1 is a side view of an egg production facility that houses laying hens with the invention/applicator attached.

FIG. 2 is an end view of an elevated roof concept that attaches to an industrial production facility.

FIG. 3 is an end view of the bottom exhausting system of the present invention in an elevated horizontal setting.

FIG. 4 is a side view of the system of FIG. 3 applicator with the elevated horizontal setting.

### DETAILED DESCRIPTION

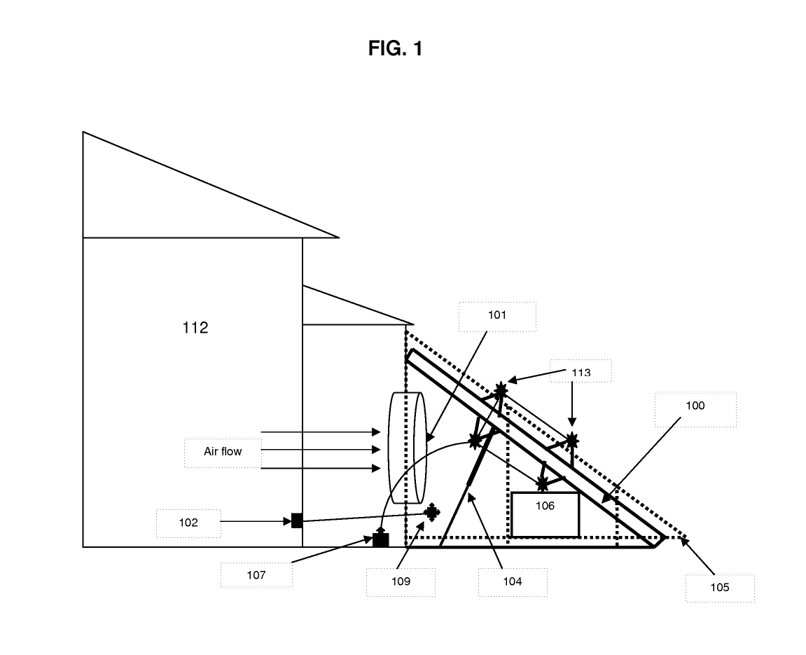

In FIG. 1 an egg production facility 112, is featured to house chickens that produce feces and said feces is contained within the production facility. Exhaust fans 101, take air from inside the production facility 112, and release the air outside the production facility 112. These fans may range from 3000 cubic feet per minute (CFM) to 25,000 CFM, however other suitable fans could be used where appropriate to the particular application.

For this applicator setting, one end of the porous media containment tray 100 is placed on the ground. The dimensions of this tray are approximately 6 foot by 9 foot by 5 inches thick and accommodate CFM ranges from 3000 CFM to 10,000 CFM. A larger dimension porous media containment tray 100, would be required for a CFM capacity above 10,000 CFM's. The other end of the porous media containment tray 100, is set in such a way as to enclose the exhaust fan 101. In some installations, the production facility's external wall may be utilized as a part of the enclosing structure. In other installations where the fan is set away from the production facility's external wall, a back panel or wall must be fabricated. The porous media containment tray 100, uses gravity to contain the media eliminating the need for two-sided walls (other than the thickness of the containment tray 100). The angle of the porous media containment tray 100, is placed in such a way as to not exceed 35 degrees and is supported by the adjustable legs 104 or suitable stationary supports. The porous media containment tray 100, is made of stainless steel or other durable material. The bottom of the porous media containment tray 100, has polyvinylchloride coated chicken wire or other suitable durable mesh material attached to it to allow air through the media but not allow the media to fall through it. The holes in the chicken wire are approximately ¾ inch squares. The enclosure walls 105, around the porous media containment tray 100, are made of ½ inch 2 sided MDO board or other suitable durable material. For exhaust fans that are set out from the production facility's external walls, an enclosing back panel will be required. An access door 106, is cut into the enclosure walls 105, to allow a human to at least partially enter and visually inspect the IPC. The exhaust fan's emissions 101, are channeled underneath the porous media containment tray 100, and then allowed to pass through the layer of organic media from the bottom/up. Spray nozzles 113, are placed above and below the porous media containment tray 100. The spray nozzles 113, are attached to the porous media containment tray 100, by use of a bracket and bolt. The spray nozzles 113, are interconnected and placed in a way to cover the entire media bed equally. The spray nozzles 113, can be 1 gallon per minute full cone nozzles or other suitable volume and pattern nozzles. The entire spraying system is then attached to a water source and hydration system controller 107. A humidity sensor may be incorporated as part of or co-located with the water source and hydration system controller 107 and used in automating the hydration system.

The pressure sensor 109, is placed inside the enclosure walls 105, and connected to a triggering device and system operation and maintenance control module 102. The air pressure tolerance level is determined and programmed into the pressure sensor 109. As dust builds up at the IPC the ventilation compromise can be monitored.

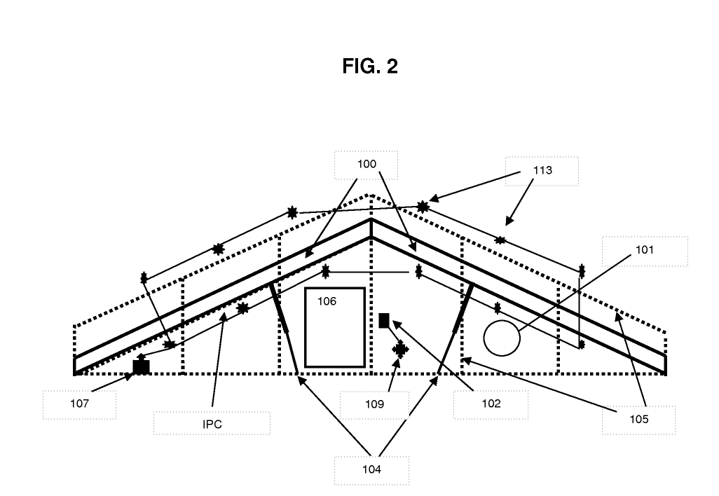

In another applicator setting, FIG. 2, two porous media containment trays 100, are placed back-to-back. The exhaust fan's emissions 101, are contained by the enclosure walls 105, and channeled through the media. The angle of the porous media containment trays 100, are placed in such a way as to not exceed 35 degrees and are supported by the adjustable legs 104 or suitable stationary support structures. An access door 106, is cut into the enclosure walls 105, to allow a human to enter and inspect the IPC. Spray nozzles 113, are placed above and below the porous media containment tray 100. The spray nozzles 113, are attached to the porous media containment tray 100, by use of a bracket and bolt. The spray nozzles 113, are interconnected and placed in a way to cover the entire media bed equally. The spray nozzles 113, can be 1 gallon per minute full cone nozzles or other suitable volume and pattern nozzles. The entire spraying system is then attached to a water source and hydration system controller 107, and may include therein and be automated with the use of humidity sensors, microprocessors, electronic valves, etc., and other means for controlling hydration.

The pressure sensor 109, is placed inside the enclosure walls 105, and connected to a triggering device and system operation and maintenance control module 102. The air pressure tolerance level is determined and programmed into the pressure sensor 109. As dust builds up at the IPC, the ventilation compromise can be monitored. If ventilation threshold tolerance is exceeded, a cleaning of the IPC is achieved by manually scraping or brushing the IPC or by manually or mechanically spraying the IPC of the porous media containment tray 100, thus relieving the ventilation compromise.

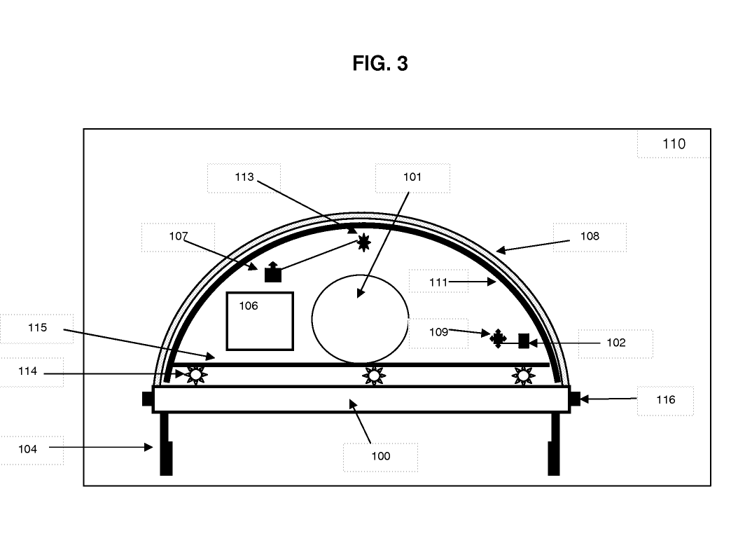

In FIG. 3, the production facility 110, may house swine. The exhaust fan 101, takes air from inside the production facility 110, and releases the air outside the production facility 110 into the filtering system. The air flow is directed by the transparent enclosing structure 108, which is made of polycarbonate or other suitable transparent material. The transparent enclosing structure 108, is supported by support beams 111, and attached to the porous media containment tray 100, by using a siding bracket 116. The exhaust air is directed downward through the porous media containment tray 100. The exhaust fans 101, may range from 3000 cubic feet per minute (CFM) to 10,000 CFM. The porous media containment tray 100, is elevated above the ground and placed horizontally with the ground and supported by the adjustable legs 104. Adjustable legs 104, allow for more flexibility in enclosing the exhaust fan. However, suitable stationary support structures may be used. The size of the applicator varies upon the volume of emissions that are passed through it and the air quality levels desired. In this application the fan CFM is 6000 and the porous media containment tray 100, is 6 foot by 12 foot by 5 inches thick. The porous media containment tray 100 is made of stainless steel or other durable material. The bottom of the porous media containment tray 100, has polyvinylchloride coated chicken wire or other suitable durable mesh material attached to it to allow air through the media but not allow the media to fall through it. The holes in the chicken wire are approximately ¾ inch squares.

Spray nozzles 113, are placed only above the porous media containment tray 100. This applicator setting creates the IPC to be on the top of the media eliminating the need for spray nozzles 113, to be placed below the porous media containment tray 100. The spray nozzles 113, are attached to the support beams 111, by use of a bracket and bolt. The spray nozzles 113, are interconnected and placed in a way to cover the entire media bed equally. The spray nozzles 113, can be 1 gallon per minute full cone nozzles or other suitable volume and pattern nozzles. The entire spraying system is then attached to a water source and hydration system controller 107.

An access door 106, is placed at the end of the applicator to allow access to the IPC. The pressure sensor 109, is placed inside the translucent and/or transparent enclosing structure 108, and connected to a triggering device and system operation and maintenance control module 102. This sensor can be preset to identify predetermined ventilation thresholds. If, for instance, a reduction in ventilation capacity of thirty percent is the triggering point, a stirring rake 114, can move across the top of the media to mechanically mix and blend the media reducing the ventilation compromise. This stirring rake 114, may spin, turn, roll, vibrate or move slowly across the media. Tracks 115, for guiding the stirring rake 114, are added to the sides of the containment tray. The media may also be manually stirred or mixed with the use of a garden rake or similar device.

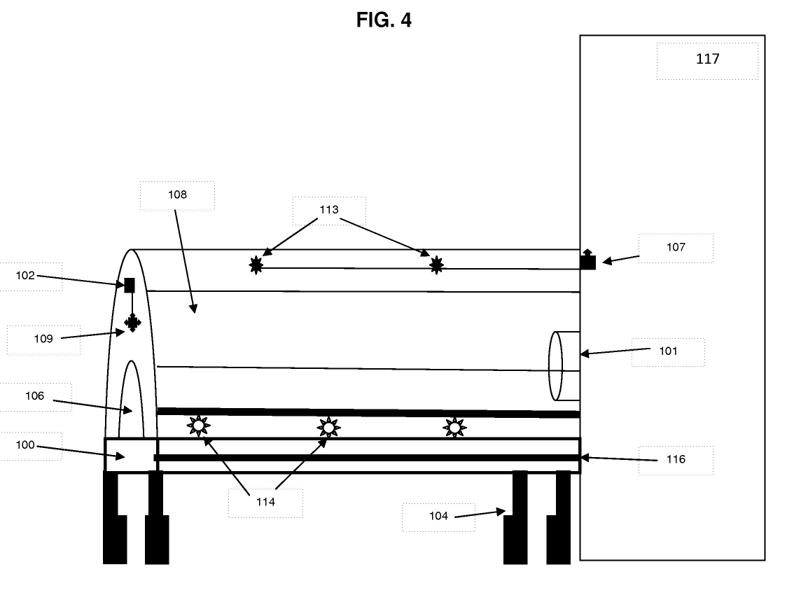

FIG. 4 is a side view of the horizontal applicator setting. The production facility 117, may house turkeys. The exhaust fan 101, takes air from inside the production facility 117, and releases the air outside the production facility 117, into the filtering system. The air flow is directed by the transparent enclosing structure 108, which is made of polycarbonate or other suitable transparent material. The transparent enclosing structure 108, is attached to the porous media containment tray 100, by using a siding bracket 116. The exhaust air is directed downward through the porous media containment tray 100. An access door 106, is placed at the end of the applicator to allow access to the IPC. The pressure sensor 109, is placed inside the transparent enclosing structure 108, and connected to a triggering device and system operation and maintenance control module 102. This sensor can be preset to identify predetermined ventilation thresholds. A stirring rake 114, can move across the top of the media to mechanically mix and blend the media reducing the ventilation compromise. This stirring rake 114, may spin, turn, roll, vibrate or move slowly across the media. Tracks 115, for guiding the stirring rake 114, are added to the sides of the containment tray. The media may also be manually stirred or mixed with the use of a garden rake or similar device.

Spray nozzles 113, are placed only above the porous media containment tray 100. This applicator setting creates the IPC to be on the top of the media eliminating the need for spray nozzles 113, to be placed below the porous media containment tray 100. The spray nozzles 113, are attached to the support beams 111, by use of a bracket and bolt. The spray nozzles 113, are interconnected and placed in a way to cover the entire media bed equally. The spray nozzles 113, can be 1 gallon per minute full cone nozzles or other suitable volume and pattern nozzles. The entire spraying system is then attached to a water source and hydration system controller 107. In every applicator setting, the operator of the system has physical access to the IPC where maintenance and inspection can easily occur.

Note that various electrical signal wires between the various components which could receive or provide information to and from the triggering device and system operation and maintenance control module 102, and a water source and hydration system controller 107, have been omitted for clarity. Such wires should be easily understood to exist where helpful. Also wireless communication and battery powered control components are not shown, but are intended to be included in any of the means for controlling hydration and/or means for controlling ventilation through the IPC.

### DEFINED TERMS

The term meant for electronically controlling physical characteristics of the initial point of contact shall specifically include any of the hydration system controller, any electrically controlled valves, humidity sensors; system operation and maintenance control module, air pressure sensor electrically controlled stirring rake or any other device or system configured to change physical characteristics of said initial point of contact; however such term shall specifically exclude any electronic or other control of an exhaust fan.

We claim:

1. A wood chip based bio-filtration system for removing matter and pollutants from an airflow exiting a confined animal feeding operation building; the system comprising:
a layer of organic media with microorganisms attached thereto, disposed in said airflow for removing odor from air passing therethrough;
said layer of organic media with microorganisms attached thereto having an initial point of contact where said air flow first encounters a portion of said layer of organic media with microorganisms attached thereto;
said initial point of contact being located so as to allow access by a human being, at least partially located within said airflow, to facilitate direct visual inspection of said initial point of contact; and
said layer of organic media with microorganisms attached thereto comprises at least one of wood chips, tree bark, peat, and straw.

2. The system of claim 1 further comprising means for controlling physical characteristics of the initial point of contact.

3. The system of claim 1 further comprising an enclosure exterior of the confined animal feeding operation building wherein said layer of organic media with microorganisms attached thereto forms a portion of said enclosure.

4. The system of claim 3 further comprising an access door in said enclosure to allow ingress of a human being into said enclosure.

5. The system of claim 1 wherein a direction of airflow through said layer of organic media with microorganisms attached thereto has a substantially upward direction.

6. The system of claim 1 wherein a substantial portion of said enclosure is formed with translucent material.

7. The system of claim 6 wherein a substantial portion of said enclosure is formed with transparent material.

8. The system of claim 1 further comprising a means for hydrating said initial point of contact, such that said airflow tends to move moisture provided by said hydration system, when said moisture first contacts said airflow, in a direction toward said initial point of contact.

9. The system of claim 1 further comprising a means for mixing and blending said layer of organic media with microorganisms attached thereto with other matter which has accumulated at the initial point of contact so as to increase a consumption rate of the microorganisms to balance an emission rate of an exhaust fan and overall increase the rate of air flowing past said initial point of contact.

10. A bio-filtration system for removing matter and pollutants from an airflow which has been exhausted from a confined animal feeding operation building; the system comprising:
an enclosure confining and channeling a portion of said airflow;
a layer of organic media with microorganisms attached thereto, disposed in said airflow for removing odor from air passing therethrough;
said layer of organic media with microorganisms attached thereto forming a portion of said enclosure through which said airflow is permitted to exit the enclosure;
said layer of organic media with microorganisms attached thereto having an initial point of contact where said air flow first encounters a portion of said layer of organic media with microorganisms attached thereto;
said enclosure being configured and said initial point of contact being located within said enclosure so as to allow a human being to perform a visual inspection said initial point of contact.

11. The system of claim 10 wherein said enclosure is configured to allow ingress of a human being into the enclosure to perform said visual inspection or maintenance.

12. The system of claim 10 further comprising means for directly hydrating said initial point of contact.

13. The system of claim 10 wherein said layer of organic media with microorganisms attached thereto comprises one of wood chips, tree bark, peat and straw.

14. The system of claim 10 further comprising an agitating means for dispersing matter which has accumulated at said point of initial contact so as to improve an overall rate of airflow through the layer of organic media.

15. The system of claim 14 wherein said agitating means further comprises an air pressure sensor for determining when said agitating means is actuated.

16. The system of claim 15 wherein said enclosure is a transparent tunnel structure disposed over said layer of organic media.

17. The system of claim 10 wherein a portion of said enclosure is the surface of the earth.

18. The system of claim 16 wherein said airflows through said layer of organic media in a substantially downward direction and where said enclosure further has an access door on an end of the enclosure opposite from the confined animal feeding operation building.

19. The system of claim 10 wherein said layer of organic media is disposed in a container which is supported on one end by the earth, at another end by an exterior portion of said confined animal feeding operation building or by a separate enclosing wall and intermediately by an adjustable leg or suitable stationary support.

20. A bio-filtration system for removing matter and pollutants from an airflow which has been exhausted from a confined bird feeding operation building; the system comprising:
a semi-cylindrical enclosure disposed adjacent to an exhaust fan of said confined bird feeding operation building, said enclosure confining a portion of said airflow;
said semi cylindrical enclosure extending orthogonally from said confined bird feeding operation building comprising:
a transparent polycarbonate roof panel over a skeletal frame;
a stainless steel containment tray configured for holding porous organic media;
a support structure to elevate said stainless steel containment tray above the surface of the earth;
an end panel, disposed opposite of the exhaust fan;
a layer of organic media with microorganisms attached thereto, disposed in said stainless steel containment tray and in said airflow for removing odor from air passing therethrough;
said layer of organic media with microorganisms attached thereto forming a portion of said enclosure through which said airflow is permitted to exit the enclosure;
said layer of organic media with microorganisms attached thereto having an initial point of contact where said air flow first encounters a portion of said layer of organic media with microorganisms attached thereto; said initial point of contact being substantially planar and parallel to the surface of the earth and on a topside of the layer of organic media;
said transparent polycarbonate roof panel being configured so as to allow a human being to perform a visual inspection of said initial point of contact from outside said enclosure;
said access door to allow a human being to physically enter the enclosure to mix the organic media at the initial point of contact;
a pressure sensor disposed in and configured for measuring air pressure within said enclosure;
said pressure sensor for providing for an actuating signal;
an automated stirring rake which is sized, configured and located so as when actuated by said actuating signal, to disperse matter accumulating at said initial point of contact which may impeded airflow through said layer of organic media and thereby increase air pressure within said enclosure;
the automated stirring rake may be configured to continue to operate until the pressure sensor determines that the air pressure within the enclosure has dropped below a termination threshold; and
a hydration system of spray nozzles disposed above the initial point of contact and spraying water thereon for maintaining a moist environment for microbial growth.
